(19) United States
(12) Patent Application Publication
(10) Pub. No.: US 2015/0134023 A1
(43) Pub. Date: May 14, 2015

(54) METHOD AND SYSTEM FOR SELECTING LV PACING SITE IN A MULTIPOLAR LV LEAD

(71) Applicant: PACESETTER, INC., SYLMAR, CA (US)

(72) Inventors: Luke McSpadden, Bologna (IT); Kyungmoo Ryu, Palmdale, CA (US); Alex Soriano, Ventura, CA (US)

(73) Assignee: PACESETTER, INC., SYLMAR, CA (US)

(21) Appl. No.: 14/080,565

(22) Filed: Nov. 14, 2013

(51) Int. Cl. A61N 1/37

(52) U.S. Cl. CPC A61N 1/3712; A61N 1/3704
USPC 607/17; 607/28

(57) ABSTRACT

A method and system for selecting at least one left ventricular (LV) pacing site for an implantable medical device equipped for cardiac stimulus pacing using a multi-pole LV lead are provided. The method and system include sensing LV activation events at multiple LV sensing sites. The arrival times of the LV activation events for corresponding LV sensing sites are measured. The method and system further include calculating differences between the arrival times for combinations of the LV sensing sites to obtain inter-site arrival delays between the combinations of the LV sensing sites. When at least one of the inter-site arrival delays exceeds a threshold, the method and system include designating the LV sensing site from the corresponding combination that has a later arrival time as a first LV pacing site from which to deliver LV pacing pulses using the implantable medical device.

### BACKGROUND

One or more embodiments of the inventive subject matter relate to selecting one or more left ventricular (LV) pacing sites in a multipolar LV lead using a measured conduction delay from pacing to sensing.

Implantable stimulation devices or cardiac pacemakers are a class of cardiac rhythm management devices that provide electrical stimulation in the form of pacing pulses to selected chambers of the heart. As the term is used herein, a pacemaker is any cardiac rhythm management device with a pacing functionality regardless of any additional functions it may perform, such as cardioversion/defibrillation.

A pacemaker is comprised of two major components, a pulse generator and a lead. The pulse generator generates the pacing stimulation pulses and includes the electronic circuitry and the power cell or battery. The lead, or leads, is implanted within the heart and has electrodes which electrically couple the pacemaker to the desired heart chamber(s). A lead may provide both unipolar and bipolar pacing and/or sensing configurations. In the unipolar configuration, the pacing pulses are applied (or responses are sensed) between an electrode carried by the lead and a case of the pulse generator or a coil electrode of another lead within the heart. In the bipolar configuration, the pacing pulses are applied (or responses are sensed) between a pair of electrodes carried by the same lead. Pacemakers are also described as single-chamber or dual-chamber systems. A single-chamber system stimulates and senses in one chamber of the heart (an atrium or a ventricle). A dual-chamber system stimulates and/or senses in at least one atrial chamber and at least one ventricular chamber. Recently, there has been the introduction of pacing systems that stimulate multiple sites in the same chamber, termed multisite stimulation systems.

When the patient's own intrinsic rhythm fails, pacemakers can deliver pacing pulses to a heart chamber to induce a depolarization of that chamber, which is followed by a mechanical contraction of that chamber. Pacemakers further include sensing circuits that sense cardiac activity for the detection of intrinsic cardiac events such as intrinsic atrial depolarizations (detectable as P waves) and intrinsic ventricular depolarizations (detectable as R waves). By monitoring cardiac activity, the pacemaker circuits are able to determine the intrinsic rhythm of the heart and provide stimulation pacing pulses that force atrial and/or ventricular depolarizations at appropriate times in the cardiac cycle when required to help stabilize the electrical rhythm of the heart. This therapy is referred to as cardiac resynchronization therapy (CRT).

Recent studies have suggested that up to 40% of patients do not respond adequately to CRT. Suboptimal LV pacing location may be responsible for many non-responders. Recent studies have shown that the site or sites of LV pacing which produce the best acute hemodynamic response varies widely from patient to patient and that the difference between best and worst pacing sites within a single patient is often significant. Therefore, patients may benefit from individualized LV pacing location(s) attempting to target one or more preferred pacing sites for each patient. Accordingly, it is important to determine individualized LV pacing sites which produce the best acute hemodynamic responses to improve clinical outcome in patients undergoing CRT.

### SUMMARY

In an embodiment, a method is provided for selecting at least one left ventricular (LV) pacing site for an implantable medical device equipped for cardiac stimulus pacing using a multi-pole LV lead. The method includes sensing LV activation events at multiple LV sensing sites. The activation events are generated in response to a delivery of a pacing pulse. The method also includes measuring arrival times of the LV activation events for the corresponding LV sensing sites. The arrival times each correspond to a conduction time from delivery of the pacing pulse until sensing of the corresponding LV activation event. The method further includes calculating differences between the arrival times for combinations of the LV sensing sites to obtain inter-site arrival delays between the combinations of the LV sensing sites. When at least one of the inter-site arrival delays exceeds a threshold, the method includes designating the LV sensing site from the corresponding combination that has a later arrival time as a first LV pacing site from which to deliver LV pacing pulses using the implantable medical device.

In an embodiment, an implantable medical device (IMD) equipped for cardiac stimulus pacing using a multi-pole left ventricular (LV) lead is provided. The IMD includes a sensor configured to sense LV activation events at multiple LV sensing sites. The activation events are generated in response to a delivery of a pacing pulse. The IMD also includes an arrival measurement (AM) module configured to measure arrival times of the LV activation events for the corresponding LV sensing sites. The arrival times each correspond to a conduction time from delivery of the pacing pulse until sensing of the corresponding LV activation event. The IMD further includes a delay calculation (DC) module configured to calculate differences between the arrival times for combinations of the LV sensing sites to obtain inter-site arrival delays between the combinations of the LV sensing sites. The IMD also includes a site designation (SD) module. When at least one of the inter-site arrival delays exceeds a threshold, the SD module is configured to designate the LV sensing site from the corresponding combination that has a later arrival time as a first LV pacing site from which to deliver LV pacing pulses using the IMD.

### BRIEF DESCRIPTION OF THE DRAWINGS

FIG. 1 illustrates an implantable stimulation device implanted into a patient's heart according to an embodiment.

FIG. 2 illustrates a block diagram of internal components of the implantable stimulation device of FIG. 1 according to an embodiment.

FIG. 3 illustrates a functional block diagram of an external device according to an embodiment.

FIG. 4 is a flow chart for a process for selecting at least one LV pacing site for an IMD equipped for cardiac stimulus pacing using a multi-pole lead according to an embodiment.

FIG. 5 displays a graph plotting multiple data streams measured in connection with different sensing sites.

FIG. 6 is a flow chart for a process for selecting at least one LV pacing vector for an IMD equipped for cardiac stimulus pacing using a multi-pole lead according to an embodiment.

### DETAILED DESCRIPTION

The systems described herein can include or represent hardware and associated instructions (e.g., software stored on a tangible and non-transitory computer readable storage medium, such as a computer hard drive, ROM, RAM, or the like) that perform the operations described herein. The hardware may include electronic circuits that include and/or are connected to one or more logic-based devices, such as microprocessors, processors, controllers, or the like. These devices may be off-the-shelf devices that perform the operations described herein from the instructions described above. Additionally or alternatively, one or more of these devices may be hard-wired with logic circuits to perform these operations.

The foregoing summary, as well as the following detailed description of certain embodiments, will be better understood when read in conjunction with the appended drawings. To the extent that the figures illustrate diagrams of the functional blocks of various embodiments, the functional blocks are not necessarily indicative of the division between hardware and circuitry. Thus, for example, one or more of the functional blocks (e.g., processors or memories) may be implemented in a single piece of hardware (e.g., a general purpose signal processor, microcontroller, random access memory, hard disk, and/or the like). Similarly, the programs may be standalone programs, may be incorporated as subroutines in an operating system, may be functions in an installed imaging software package, and the like. Furthermore, to the extent that the figures illustrate flow diagrams of processes of various embodiments, the operations may be described by adding, rearranging, combining, or omitting the illustrated operations without departing from the scope of the processes as described herein. It should be understood that the various embodiments are not limited to the arrangements and instrumentality shown in the drawings.

The block diagrams of embodiments herein illustrate various blocks labeled “module”. It is to be understood that the modules represent circuit modules that may be implemented as hardware with associated instructions (e.g., software stored on a tangible and non-transitory computer readable storage medium, such as a computer hard drive, ROM, RAM, or the like) that perform the operations described herein. The hardware may include state machine circuitry hard-wired to perform the functions described herein. Optionally, the hardware may include electronic circuits that include and/or are connected to one or more logic-based devices, such as microprocessors, processors, controllers, or the like. Optionally, the modules may represent processing circuitry such as one or more field programmable gate array (FPGA), application specific integrated circuit (ASIC), or microprocessor. The circuit modules in various embodiments may be configured to execute one or more algorithms to perform functions described herein. The one or more algorithms may include aspects of embodiments disclosed herein, whether or not expressly identified in a flowchart or a method.

As used herein, an element or step recited in the singular and proceeded with the word “a” or “an” should be understood as not excluding plural of said elements or steps, unless such exclusion is explicitly stated. Furthermore, references to “an embodiment” or “one embodiment” of the inventive subject matter are not intended to be interpreted as excluding the existence of additional embodiments that also incorporate the recited features. Moreover, unless explicitly stated to the contrary, embodiments “including,” “comprising,” or “having” (and various forms thereof) an element or a plurality of elements having a particular property may include additional such elements not having that property.

One or more embodiments generally relate to implantable medical devices and systems such as pacemakers and implantable cardioverter-defibrillators (ICDs). One or more embodiments relate, in particular, to such devices and systems that include a multi-pole LV lead capable of pacing from one or more electrodes along the multi-pole lead, and methods for use therewith.

New multipolar left ventricular (LV) leads have been developed for implantable medical devices (IMDs) that include multiple electrodes for placement in the LV chamber. For example, St. Jude Medical, Inc. (headquartered in St. Paul, Minn.) has developed the Quartet™ LV pacing lead, which includes four pacing electrodes on the LV lead—enabling up to 10 pacing configurations (e.g., pacing vectors). The multipolar leads allow for the possibility of “electronic repositioning”—the ability to non-invasively change the site of LV pacing simply by programming pacing with a different LV electrode on the same or a different multipolar lead. Furthermore, a more detailed picture of the activation sequence along the lead body is available with multipolar LV leads than with unipolar or bipolar leads.

Data from recent studies has suggested that pacing from a site of late activation produces a better acute hemodynamic response than pacing from an early site. An activation or activation event is a detected response to a pacing pulse along a sensing vector. The better acute hemodynamic response can result in an improved clinical outcome in patients undergoing CRT. In one or more embodiments described herein, an IMD with a multipolar LV lead is used to measure LV activation times along the multipolar LV lead after RV pacing. The measured LV activation times are used to identify and automatically program/reprogram to a preferred site for LV pacing that may result in improved acute hemodynamic response.

An embodiment describes a process to select and automatically program/reprogram to a preferred LV pacing site in a multipolar LV lead based on RV pace to LV sense conduction delay measurements. The process may be performed at least in part by an algorithm within the IMD or an external programmer. The conduction delay measurements refer to the conduction time from delivery of the pacing pulse until sensing of the corresponding LV activation event at various LV sensing sites in response to the pacing pulse, and may also be referred to herein as “arrival time.” Patient data has shown that when the relative conduction delay between two sites (e.g., inter-site arrival delay or Δdelay) is greater than a threshold (e.g., 10 ms), acute hemodynamic response (e.g., dP/dt_{Max}) may be significantly greater when pacing at the later activating site. The later activating site refers to the LV sensing site that had a greater arrival time. Pacing at the later activating site refers to using the LV electrode at the LV sensing site as a pacing cathode to deliver pacing pulses to the heart.

The algorithm may select a preferred site when an inter-site arrival delay is greater than the threshold, with the preferred site being the later activating LV sensing site. The algorithm that performs the process may recommend the selected preferred site to a physician in-clinic or, if out-of-clinic, automatically program/reprogram to the preferred site. Automatic programming to the preferred site may include the IMD electronically repositioning the multipolar lead to pace at the preferred site. By pacing from at least the determined preferred site, CRT may produce a better hemodynamic response than when pacing from non-preferred sites. If, for example, none of the calculated inter-site arrival delays exceed the threshold, there is no significant benefit gained by pacing at the later site, so neither LV electrode is preferable over the other based solely on LV activation arrival time. As such, a previous pacing site may remain unchanged or the pacing site may be selected based on other criteria (e.g., lower capture threshold of an available pacing vector, lack of phrenic nerve stimulation, etc.).

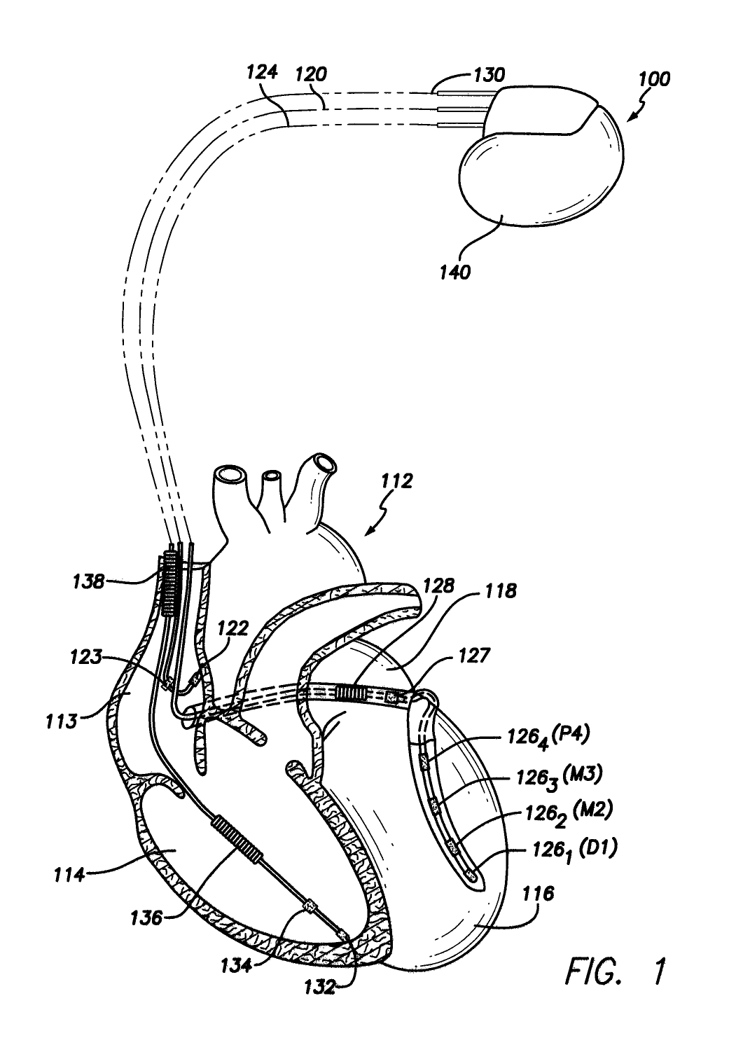

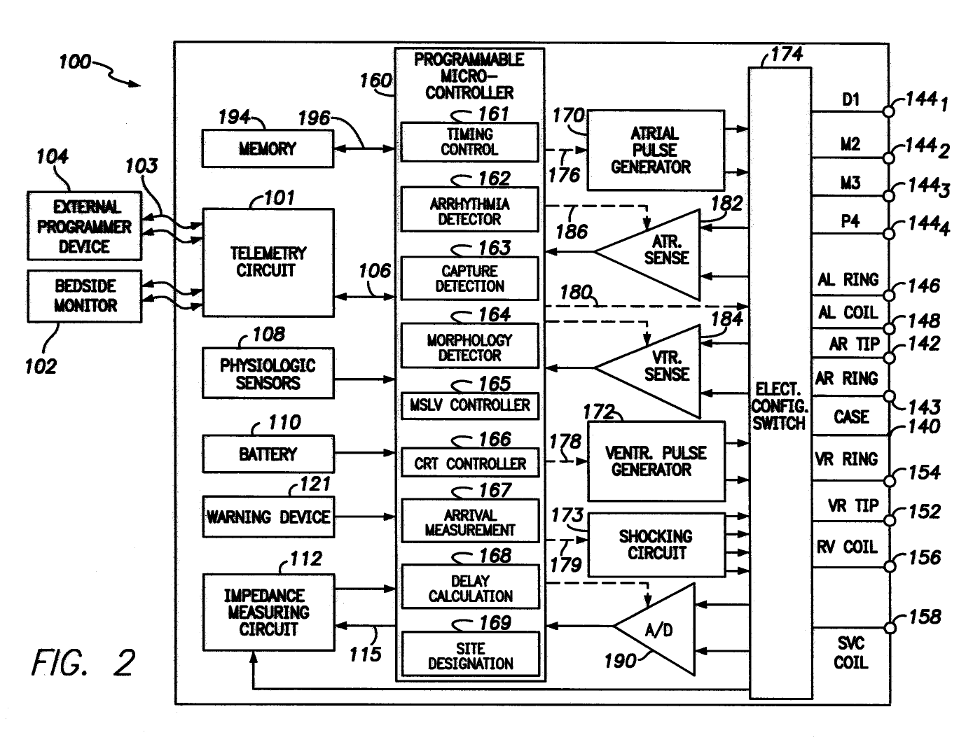

In view of the above, FIGS. 1 and 2 illustrate an IMD equipped for cardiac stimulus pacing using a multi-pole LV lead, in which embodiments described herein may be implemented.

FIG. 1 illustrates an implantable medical device (IMD) 100 in electrical communication with multiple leads implanted into a patient's heart 112 for delivering multi-chamber stimulation and sensing cardiac activity according to an embodiment. The IMD 100 may be a dual-chamber stimulation device, including a pacemaker/ICD, capable of treating both fast and slow arrhythmias with stimulation therapy, including cardioversion, defibrillation, and pacing stimulation, including CRT. Optionally, the IMD 100 may be configured for multi-site left ventricular (MSLV) pacing, which provides pacing pulses at more than one site within the LV chamber each pacing cycle. The IMD 100 may be referred to herein as pacemaker/ICD 100. To provide atrial chamber pacing stimulation and sensing, pacemaker/ICD 100 is shown in electrical communication with a heart 112 by way of a left atrial (LA) lead 120 having an atrial tip electrode 122 and an atrial ring electrode 123 implanted in the atrial appendage 113. Pacemaker/ICD 100 is also in electrical communication with the heart 112 by way of a right ventricular (RV) lead 130 having, in this embodiment, a ventricular tip electrode 132, an RV ring electrode 134, an RV coil electrode 136, and a superior vena cava (SVC) coil electrode 138. The RV lead 130 is transvenously inserted into the heart 112 so as to place the RV coil electrode 136 in the RV apex, and the SVC coil electrode 138 in the superior vena cava. Accordingly, the RV lead 130 is capable of receiving cardiac signals and delivering stimulation in the form of pacing and shock therapy to the right ventricle 114 (also referred to as the RV chamber).

To sense left atrial and ventricular cardiac signals and to provide left ventricle 116 (e.g., left chamber) pacing therapy, pacemaker/ICD 100 is coupled to a multi-pole LV lead 124 designed for placement in the “CS region.” As used herein, the phrase “CS region” refers to the venous vasculature of the left ventricle, including any portion of the coronary sinus (CS), great cardiac vein, left marginal vein, left posterior ventricular vein, middle cardiac vein, and/or small cardiac vein or any other cardiac vein accessible by the coronary sinus. In an embodiment, an LV lead 124 is designed to receive atrial and ventricular cardiac signals and to deliver left ventricular pacing therapy using a set of multiple LV electrodes 126 that includes electrodes 126₁, 126₂, 126₃, and 126₄ (thereby providing a multipolar or multi-pole lead). The LV lead 124 also may deliver left atrial pacing therapy using at least an LA ring electrode 127 and shocking therapy using at least an LA coil electrode 128. In alternate embodiments, the LV lead 124 includes the LV electrodes 126₁, 126₂, 126₃, and 126₄, but does not include the LA electrodes 127 and 128. The LV lead 124 may be, for example, the Quartet™ LV pacing lead developed by St. Jude Medical Inc. (headquartered in St. Paul, Minn.), which includes four pacing electrodes on the LV lead—enabling up to 10 pacing vectors or configurations. Although three leads 120, 124, and 130 are shown in FIG. 1, fewer or additional leads with various numbers of pacing, sensing, and/or shocking electrodes may optionally be used. For example, the LV lead 124 may have more or less than four LV electrodes 126.

The LV electrode 126₁ is shown as being the most “distal” LV electrode with reference to how far the electrode is from the left atrium 118. The LV electrode 126₄ is shown as being the most “proximal” LV electrode 126 to the left atrium 118. The LV electrodes 126₂ and 126₃ are shown as being “middle” LV electrodes, between the distal and proximal LV electrodes 126₁ and 126₄, respectively. Accordingly, so as to more aptly describe their relative locations, the LV electrodes 126₁, 126₂, 126₃, and 126₄ may be referred to respectively as electrodes D1, M2, M3, and P4 (where “D” stands for “distal”, “M” stands for “middle”, and “P” stands from “proximal”, and the numbers are arranged from most distal to most proximal, as shown in FIG. 1). Optionally, more or fewer LV electrodes may be provided on the lead 124 than the four LV electrodes D1, M2, M3, and P4.

The LV electrodes 126 are configured such that each electrode may be utilized to deliver pacing pulses and/or sense pacing pulses (e.g., monitor the response of the LV tissue to a pacing pulse). In a pacing vector or a sensing vector, each LV electrode 126 may be controlled to function as a cathode (negative electrode) or an anode (positive electrode). Pacing pulses may be directionally provided between at least two electrodes to define a pacing vector. In a pacing vector, a generated pulse is applied to the surrounding myocardial tissue through the cathode. The electrodes that define the pacing vectors may be electrodes in the heart 112 or located externally to the heart 112 (e.g., on a housing/case device 140). For example, the housing/case 140 may be referred to as CAN 140 and function as an anode in unipolar pacing and/or sensing vectors. The LV electrodes 126 may be used to provide various different pacing vectors. Some of the vectors are intraventricular LV vectors (e.g., vectors between two of the LV electrodes 126), while other vectors are interventricular vectors (e.g. vectors between an LV electrode 126 and the RV coil 136 or another electrode remote from the left ventricle 116). Below is a list of exemplary bipolar vectors with LV cathodes that may be used for pacing and/or sensing using the LV electrodes D1, M2, M3, and P4 and the RV coil 136. In the following list, the electrode to the left of the arrow is assumed to be the cathode, and the electrode to the right of the arrow is assumed to be the anode.

D1→RV coil

M2→RV coil

M3→RV coil

P4→RV coil

D1→M2

D1→P4

M2→P4

M3→M2

M3→P4

P4→M2

It is noted that the preceding list is only a subset of the available pacing and sensing vectors for use with the IMD 100. For example, the LV electrodes may be used in unipolar pacing and/or sensing vectors, including D1→CAN 140, M2→CAN 140, M3→CAN 140, and P4→CAN 140. Furthermore, pacing pulses may be generated in other chambers of the heart, such as the right ventricle. For example, pacing vectors may be RV tip 132 to RV coil 136, RV ring 134 to RV coil 136, RV tip 132 to CAN 140, and the like. In one or more embodiments described herein, pacing pulses along RV pacing vectors may be sensed by unipolar or bipolar LV sensing vectors, with the recorded responses being used to determine preferred LV pacing sites.

FIG. 2 illustrates a simplified block diagram of internal components of the IMD 100 (e.g., pacemaker/ICD) according to an embodiment. While a particular pacemaker/ICD 100 is shown, it is for illustration purposes only. One of skill in the art could readily duplicate, eliminate, or disable the appropriate circuitry in any desired combination to provide a device capable of treating the appropriate chamber(s) with cardioversion, defibrillation, and pacing stimulation. The housing/CAN 140 for pacemaker/ICD 100, shown schematically in FIG. 2 may be programmably selected to act as the anode for at least some unipolar modes. The CAN 140 may further be used as a return electrode alone or in combination with one or more of the coil electrodes 128, 136, and 138 (all shown in FIG. 1) for shocking purposes.

The IMD 100 further includes a connector (not shown) having a plurality of terminals, 142, 143, 144₁-144₄, 146, 148, 152, 154, 156, and 158 (shown schematically and, for convenience, with the names of the electrodes to which they are connected). As such, to achieve right atrial (RA) sensing and pacing, the connector includes at least an RA tip terminal (A_{R} TIP) 142 adapted for connection to the atrial tip electrode 122 (shown in FIG. 1) and an RA ring (A_{R} RING) electrode 143 adapted for connection to the RA ring electrode 123 (shown in FIG. 1). To achieve left chamber sensing, pacing, and shocking, the connector includes an LV tip terminal 144₁ adapted for connection to the D1 electrode and additional LV electrode terminals 144₂, 144₃, and 144₄ adapted for connection to the M2, M3, and P4 electrodes, respectively, of the quadripolar LV lead 124 (shown in FIG. 1). The connector also includes an LA ring terminal (A_{L} RING) 146 and an LA shocking terminal (A_{L} COIL) 148, which are adapted for connection to the LA ring electrode 127 (shown in FIG. 1) and the LA coil electrode 128 (shown in FIG. 1), respectively. To support right chamber sensing, pacing, and shocking, the connector further includes an RV tip terminal (V_{R} TIP) 152, an RV ring terminal (V_{R} RING) 154, an RV coil terminal (RV COIL) 156, and an SVC coil terminal (SVC COIL) 158, which are adapted for connection to the RV tip electrode 132, the RV ring electrode 134, the RV coil electrode 136, and the SVC coil electrode 138 (all four electrodes shown in FIG. 1), respectively.

At the core of the IMD 100 is a programmable microcontroller 160, which controls the various modes of stimulation therapy. The microcontroller 160 (also referred to herein as a control unit or controller) includes a microprocessor or equivalent control circuitry, designed specifically for controlling the delivery of stimulation therapy. The microcontroller 160 may further include RAM or ROM memory, logic and timing circuitry, state machine circuitry, and/or I/O circuitry. The microcontroller 160 includes the ability to process or monitor input signals (data) as controlled by a program code stored in a designated block of memory. The details of the design and operation of the microcontroller 160 are not critical to the invention. Rather, any suitable microcontroller 160 may be used that carries out the functions described herein. Among other things, the microcontroller 160 receives, processes, and manages storage of digitized cardiac data sets from the various sensors and electrodes. For example, the cardiac data sets may include DI data, SI data, LTV information, STV information, IEGM data, pressure data, heart sound data, and the like.

An atrial pulse generator 170 and a ventricular pulse generator 172 are configured to generate and deliver a pacing pulse from at least one RV or RA pacing site, such as at one or more pacing sites along the RA lead 120, the RV lead 130, and/or the LV lead 124 (all three leads shown in FIG. 1). For example, the atrial pulse generator 170 generates pulses for delivery by the RA lead 120, while the ventricular pulse generator 172 generates pulses for delivery by the RV lead 130 and/or the LV lead 124. The pacing pulses are routed from the pulse generators 170, 172 to selected electrodes within the leads 120, 124, 130 through an electrode configuration switch 174. It is understood that in order to provide stimulation therapy in each of the four chambers of the heart, the atrial and ventricular pulse generators 170 and 172, may include dedicated, independent pulse generators, multiplexed pulse generators, or shared pulse generators. The pulse generators 170, 172 are controlled by the microcontroller 160 via appropriate control signals 176, 178, respectively, to trigger or inhibit the stimulation pulses, including the timing and output of the pulses.

The electrode configuration switch 174 may include a plurality of switches for connecting the desired electrodes to the appropriate I/O circuits, thereby providing complete electrode programmability. Accordingly, the switch 174, in response to a control signal 180 from the microcontroller 160, controls the polarity of the stimulation pulses (e.g., unipolar, bipolar, combipolar, etc.) by selectively actuating the appropriate combination of switches (not shown) as is known in the art. The switch 174 also switches among the various LV electrodes 126 to select the channels (e.g., vectors) to deliver and/or sense one or more of the pacing pulses.

Atrial sensors or sensing circuits 182 and ventricular sensors or sensing circuits 184 may also be selectively coupled to the RA lead 120, the LV lead 124, and/or the RV lead 130 (all three leads shown in FIG. 1) through the switch 174. The atrial and ventricular sensors 182 and 184 have the ability to detect the presence of cardiac activity in each of the four chambers of the heart 112 (shown in FIG. 1). For example, the ventricular sensor 184 is configured to sense LV activation events at multiple LV sensing sites, where the activation events are generated in response to a pacing pulse. In an embodiment, the ventricular sensor 184 senses along at least four sensing vectors, each sensing vector utilizing a sensing electrode in the left ventricle.

The atrial sensing circuits 182 and ventricular sensing circuits 184 may include dedicated sense amplifiers, multiplexed amplifiers, or shared amplifiers. The switch 174 determines the “sensing polarity” or sensing vector of the cardiac signal by selectively opening and/or closing the appropriate switches, as is known in the art. In this way, a clinician may program the sensing polarity independent of the stimulation polarity. The outputs of the atrial and ventricular sensing circuits 182 and 184 are connected to the microcontroller 160. The outputs, in turn, are able to trigger or inhibit the atrial and ventricular pulse generators 170 and 172, respectively, in a demand fashion in response to the absence or presence of cardiac activity in the appropriate chambers of the heart 112.

Cardiac signals are also applied to the inputs of an analog-to-digital (A/D) data acquisition system 190. The A/D data acquisition system 190 is configured to acquire intracardiac electrogram (IEGM) signals, convert the raw analog data into a digital signal, and store the digital signals for later processing and/or telemetric transmission. The telemetric transmission may be to an external programmer 104, a bedside monitor, and/or a personal advisory module (PAM) 102. The data acquisition system 190 may be operatively coupled to the RA lead 120, the LV lead 124, and the RV lead 130 (all three leads shown in FIG. 1) through the switch 174 to sample cardiac signals across any pair of desired electrodes.

The microcontroller 160 includes timing control module 161 to control the timing of the stimulation pacing pulses, including, but not limited to, pacing rate, atrio-ventricular delay, interatrial conduction delay, interventricular conduction delay, and/or intraventricular delay. The timing control module 161 can also keep track of the timing of refractory periods, blanking intervals, noise detection windows, evoked response detection windows, alert intervals, marker channel timing, etc., which is known in the art.

The microcontroller 160 further includes an arrhythmia detector 162 for operating the system 100 as an implantable cardioverter/defibrillator device. The detector 162 determines desirable times to administer various therapies. For example, the detector 162 may detect the occurrence of an arrhythmia and automatically control the application of an appropriate electrical shock therapy to the heart aimed at terminating the detected arrhythmia. To this end, the microcontroller 160 further controls a shocking circuit 173 by way of a control signal 179. The shocking circuit 173 generates shocking pulses that are applied to the heart of the patient through at least two shocking electrodes. The shocking pulses may be selected from the LA coil electrode 128, the RV coil electrode 136, and/or the SVC coil electrode 138 (all three electrodes shown in FIG. 1). The CAN 140 may act as an active electrode in combination with the RV coil electrode 136, or as part of a split electrical vector using the SVC coil electrode 138 or the LA coil electrode 128 (e.g., with the RV coil electrode 136 as a common electrode).

The microcontroller 160 may additionally include a morphology detector 164 and an MSLV controller 165. The MSLV controller 165 controls multi-site LV pacing therapy, which can be performed in conjunction with CRT pacing. As an example, the MSLV controller 165 may control the ventricular pulse generator 172 to simultaneously deliver four pacing pulses over selected corresponding pacing vectors. The arrhythmia detector 162, morphology detector 164, and/or MSLV controller 165 may be implemented in hardware as part of the microcontroller 160, or as software/firmware instructions programmed into the system 100 and executed on the microcontroller 160 during certain modes of operation.

A CRT controller 166 within the microcontroller 160 controls the actual delivery of CRT pacing pulses to synchronize the contractions of the right and left ventricles. The CRT controller 166 controls the number, timing, and output of the CRT pacing pulses delivered during each cardiac cycle, as well as over which pacing vectors the pacing pulses are to be delivered. The CRT controller 166 also selects the sensing channels over which the responses to the pulses are detected. The sensing channels or vectors are associated with corresponding pacing vectors. Immediately after pacing, the electrodes at the LV sensing sites that define the selected sensing channels monitor the LV tissue for a sensed activation event.

The microcontroller 160 further includes a local capture detection module 163. The capture detection module 163 may aid in acquisition, analysis, etc., of data streams relating to evoked responses sensed at various LV sensing sites along corresponding sensing channels. In particular, the capture detection module 163 may act to distinguish local capture versus non-capture versus undesired fusion of pacing pulses delivered along corresponding pacing vectors. The capture detection module 163 may communicate with the MSLV controller 165 and/or the CRT controller 166 to determine capture thresholds of individual pacing vectors associated with one or more LV sensing sites. The capture threshold may be used by the microcontroller 160 to determine the LV pacing site and the pacing vector at the LV pacing site along which to deliver LV pacing pulses, as described further below.

In an embodiment, the microcontroller 160 includes an arrival measurement (AM) module 167, a delay calculation (DC) module 168, and a site designation (SD) module 169. For example, when determining a preferred LV pacing site, as further described herein, the AM module 161 may be configured to measure arrival times of LV activation events for corresponding LV sensing sites. The arrival times each correspond to a conduction time from delivery of the pacing pulse until sensing of the corresponding LV activation event.

The DC module 168 is configured to calculate differences between arrival times for combinations of LV sensing sites to obtain inter-site arrival delays between the combinations of the LV sensing sites. For example, the DC module 168 may calculate a relative inter-site arrival delay between any pair of the LV sensing sites. In an embodiment, the DC module 168 may calculate the differences between an arrival time at each of the LV sensing sites and an arrival time at an LV sensing site previously designated as a current LV pacing site from which one or more LV pacing pulses have been delivered. For example, the DC module 168 may perform this function in an out-of-clinic setting when the IMD 100 automatically tests to determine whether the current LV pacing site is still preferable, or if one or more other LV sites would be more preferable than the current LV pacing site. In an embodiment, the DC module 168 may calculate the differences between an arrival time at each of the LV sensing sites and the earliest measured arrival time of an LV activation event generated in response to the pacing pulse. The DC module 168 may perform this function in a clinic setting, for example, so a clinician may determine whether the arrival time at any of the LV sensing sites exceeds the earliest measured arrival time by a threshold amount. If so, the one or more LV sensing sites may be designated as preferred candidates for delivering future LV pacing pulses, as discussed below.

The SD module 169 is configured to, when at least one inter-site arrival delay exceeds a threshold, designate the LV sensing site from the corresponding combination that has a later arrival time as a first LV pacing site from which to deliver LV pacing pulses. For example, if an arrival delay between LV electrodes M3 and P4 exceeds the threshold, then the SD module 169 would designate the electrode with the later arrival time of the pair as the first LV pacing site. Once the SD module 169 designates the first LV pacing site, the pulse generator 172 may be configured to deliver a pacing pulse or sequence from the first LV pacing site. Even if the pulse generator 172 is configured to deliver a pacing sequence from multiple LV pacing sites, a first LV pacing pulse in the sequence may be delivered from the first LV pacing site designated by the SD module 169.

The SD module 169 may also be configured to designate an LV pacing site when none of the inter-site arrival delays exceeds the threshold. For example, when none of the inter-site arrival delays exceeds the threshold, the SD module 169 may retain a previously designated LV pacing site as unchanged from which to deliver LV pacing pulses. Optionally, the SD module 169 may designate an LV sensing site having a pacing vector with a lowest capture threshold as the first LV pacing site, due to energy conservation. In order to determine relative capture thresholds of pacing vectors at LV sensing sites, the SD module 169 may obtain a capture threshold of at least one pacing vector at each LV sensing site. Optionally, the SD module 169 may designate one or more pacing vectors that have a capture threshold above a predetermined value as non-pacing vectors. The non-pacing vectors are not to be used as pacing vectors at the first LV pacing site due to the energy expense necessary to achieve local capture.

Depending upon the implementation, the aforementioned components of the microcontroller 160 may be implemented in hardware as part of the microcontroller 160, or as software/firmware instructions programmed into the device and executed on the microcontroller 160 during certain modes of operation. In addition, the modules may be separate software modules or combined to permit a single module to perform multiple functions. For example, the AM module 167 and the DC module 168 may be combined into the same module. In addition, although shown as being components of the microcontroller 160, some or all of the components/modules described above may be implemented separately from the microcontroller 160 using application specific integrated circuits (ASICs) or the like.

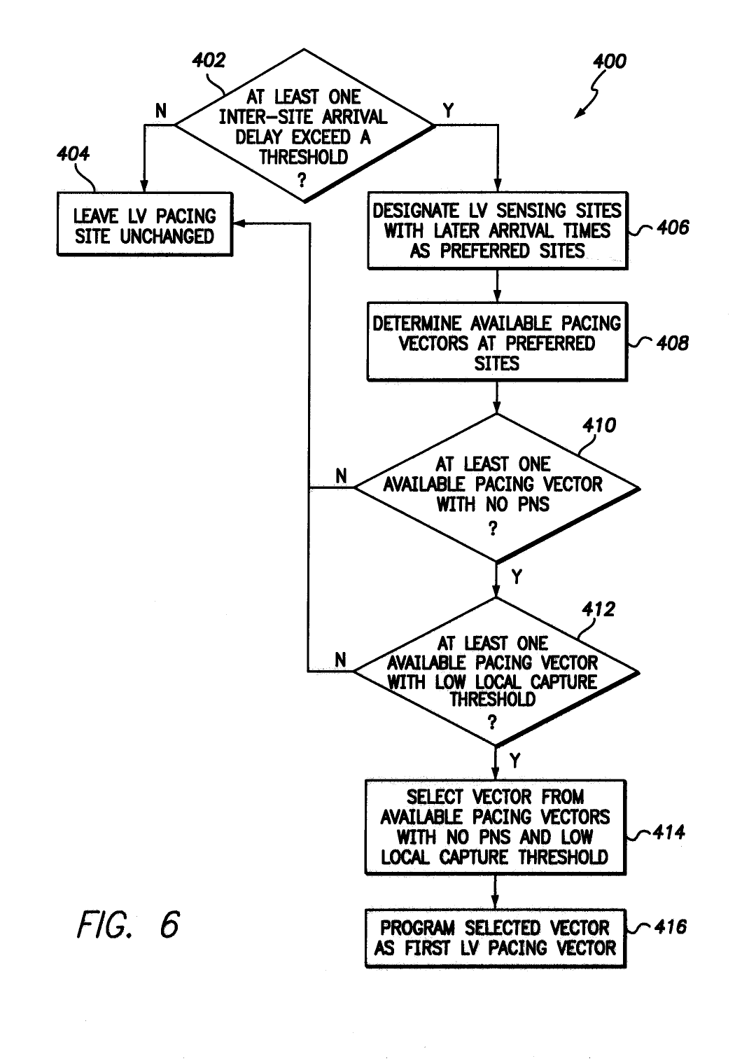

The microcontroller 160 is further coupled to a memory 194 by a suitable data/address bus 196. The programmable operating parameters used by the microcontroller 160 are stored in the memory 194 and modified, as required, in order to customize the operation of pacemaker/ICD 100 to suit the needs of a particular patient. Such operating parameters define, for example, the amplitude or magnitude of the generated pacing pulses, wave shape, pulse duration, and/or vector (e.g., including electrode polarity) for the pacing pulses. Other pacing parameters may include base rate, rest rate, and/or circadian base rate. The memory 194 also may be utilized to store, at least temporarily, determined characteristics about one or more pacing vectors, such as local capture thresholds and the presence or absence of phrenic nerve stimulation (PNS), which is a potential side effect described with reference to FIG. 6.

Optionally, the operating parameters of the implantable pacemaker/ICD 100 may be non-invasively programmed into the memory 194 through a telemetry circuit 101 in telemetric communication with an external programmer device 104 or a bedside monitor 102, such as a programmer, trans-telephonic transceiver, or a diagnostic system analyzer. The telemetry circuit 101 is activated by the microcontroller 160 through a control signal 106. The telemetry circuit 101 may allow IEGMs and status information relating to the operation of pacemaker/ICD 100 (contained in the microcontroller 160 or the memory 194) to be sent to the external device 102, and vice-versa, through an established communication link 103. An internal warning device 121 may be provided for generating perceptible warning signals to a patient and/or caregiver via vibration, voltage, or other methods.

Pacemaker/ICD 100 further includes an accelerometer or other physiologic sensor 108. The physiologic sensor 108 is commonly referred to as a “rate-responsive” sensor because it may be used to adjust the pacing stimulation rate according to the exercise state (e.g., heart rate) of the patient. However, the physiological sensor 108 may further be used to detect changes in cardiac output, changes in the physiological condition of the heart, and/or diurnal changes in activity (e.g., detecting sleep and wake states and arousal from sleep). Accordingly, the microcontroller 160 may respond to such changes by adjusting the various pacing parameters (such as rate, interatrial delay, interventricular delay, etc.) at which the atrial and ventricular pulse generators 170 and 172 generate stimulation pulses. While shown as being included within pacemaker/ICD 100, it is to be understood that the physiologic sensor 108 may also be external to the pacemaker/ICD 100. Optionally, the physiologic sensor 108 may still be implanted within or carried by the patient. A common type of rate responsive sensor 108 is an activity sensor incorporating an accelerometer or a piezoelectric crystal, which is mounted within the housing/case 140 of pacemaker/ICD 100. Other types of physiologic sensors 108 are also known, such as sensors that sense the oxygen content of blood, respiration rate and/or minute ventilation, pH of blood, ventricular gradient, stroke volume, cardiac output, contractility, and the like.

The pacemaker/ICD 100 additionally includes a battery 110, which provides operating power to all of the circuits shown in FIG. 2. The makeup of the battery 110 may vary depending on the capabilities of pacemaker/ICD 100. If the system only provides low voltage therapy (e.g., for repetitive pacing pulses), a lithium iodine or lithium copper fluoride cell may be utilized. For a pacemaker/ICD that employs shocking therapy, the battery may be configured to be capable of operating at low current drains for long periods and then providing high-current pulses (for capacitor charging) when the patient requires a shock pulse. The battery 110 may also be configured to have a predictable discharge characteristic so that elective replacement time can be detected.

As further shown in FIG. 2, the pacemaker/ICD 100 has an impedance measuring circuit 112, which is enabled by the microcontroller 160 via a control signal 115. Uses for an impedance measuring circuit 112 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. The impedance measuring circuit 112 is coupled to the switch 174 so that any desired electrode may be used.

The above described implantable medical device 100 was described as an exemplary pacemaker/ICD. One of ordinary skill in the art would understand that one or more embodiments herein may be used with alternative types of implantable devices. Accordingly, embodiments should not be limited to using only the above described device 100.

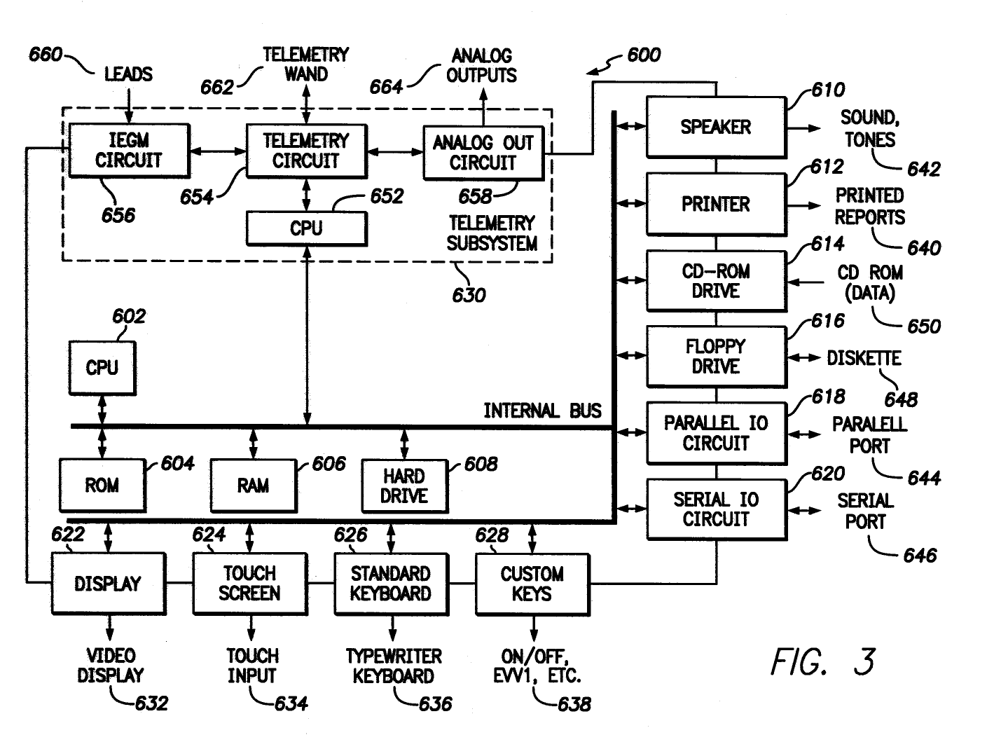

FIG. 3 illustrates a functional block diagram of an external device 600 that is operated in accordance with the processes described herein and to interface with the implantable medical device 100 as shown in FIGS. 1 and 2 and described herein. The external device 600 may be the external programmer device 104 shown in FIG. 2. The external device 600 may take the form of a workstation, a portable computer, an IMD programmer, a PDA, a cell phone, and the like. The external device 600 includes an internal bus that connects/interfaces with a Central Processing Unit (CPU) 602, ROM 604, RAM 606, a hard drive 608, a speaker 610, a printer 612, a CD-ROM drive 614, a floppy drive 616, a parallel I/O circuit 618, a serial I/O circuit 620, a display 622, a touch screen 624, a standard keyboard 626, custom keys 628, and/or a telemetry subsystem 630. The internal bus is an address/data bus that transfers information between the various components described herein. The hard drive 608 may store operational programs as well as data, such as waveform templates, determinations on presence of PNS at various electrode locations, and/or capture thresholds for pacing vectors.

The CPU 602 includes a microprocessor, a micro-controller, and/or equivalent control circuitry, designed specifically to control interfacing with the external device 600 and with the IMD 100. The CPU 602 may include RAM or ROM memory, logic and timing circuitry, state machine circuitry, and/or I/O circuitry to interface with the IMD 100.

The display 622 may be connected to a video display 632. The display 622 displays various forms of information related to the processes described herein. The touch screen 624 may display graphic user information relating to the IMD 100. The touch screen 624 accepts a user's touch input 634 when selections are made. The keyboard 626 (e.g., a typewriter keyboard 636) allows a user to enter data to displayed fields, as well as interface with the telemetry subsystem 630. Furthermore, custom keys 628 turn on/off 638 (e.g., EVVI) the external device 600. The printer 612 prints copies of reports 640 for a physician to review or to be placed in a patient file, and speaker 610 provides an audible warning (e.g., sounds and tones 642) to the user. The parallel I/O circuit 618 interfaces with a parallel port 644. The serial I/O circuit 620 interfaces with a serial port 646. The floppy drive 616 accepts diskettes 648. Optionally, the floppy drive 616 may include a USB port or other interface capable of communicating with a USB device such as a flash memory stick. The CD-ROM drive 614 accepts CD ROMs 650. The CD-ROM drive 614 optionally may include a DVD port capable of reading and/or writing DVDs.

The telemetry subsystem 630 includes a central processing unit (CPU) 652 in electrical communication with a telemetry circuit 654, which communicates with both an IEGM circuit 656 and an analog out circuit 658. The IEGM circuit 656 may be connected to leads 660. The IEGM circuit 656 is also connected to the implantable leads 120, 124 and 130 (shown in FIG. 1) to receive and process IEGM cardiac signals. Optionally, the IEGM cardiac signals sensed by the leads 120, 124 and 130 may be collected by the IMD 100 and then wirelessly transmitted to the telemetry subsystem 630 input of the external device 600.

The telemetry circuit 654 is connected to a telemetry wand 662. The analog out circuit 658 includes communication circuits to communicate with analog outputs 664. The external device 600 may wirelessly communicate with the IMD 100 and utilize protocols, such as Bluetooth, GSM, infrared wireless LANs, HIPERLAN, 3G, 4G, satellite, as well as circuit and packet data protocols, and the like. Alternatively, a hard-wired connection may be used to connect the external device 600 to the IMD 100.

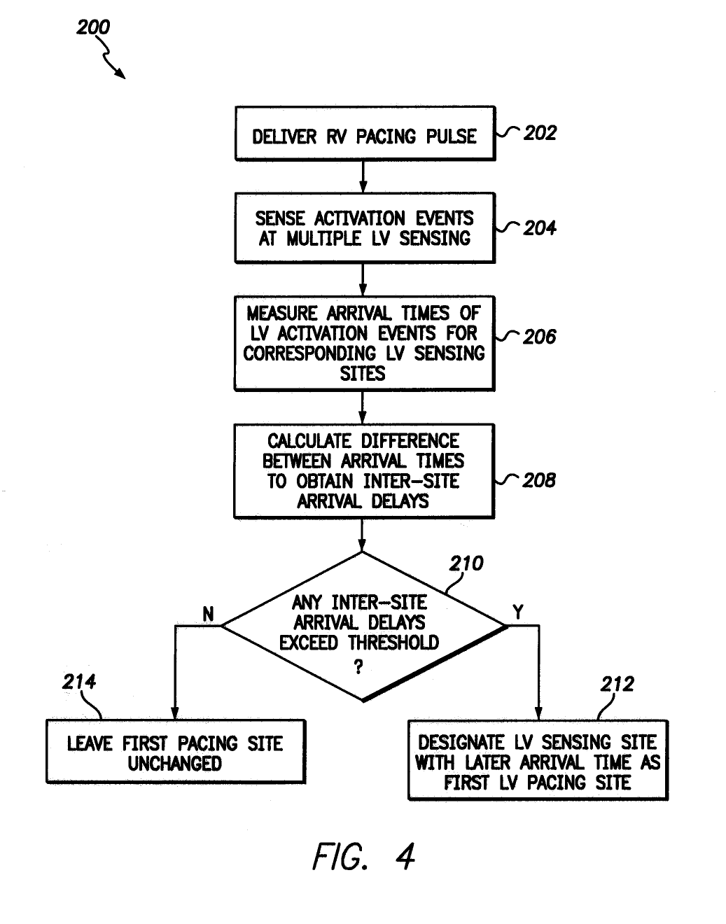

FIG. 4 is a flow chart for a process 200 for selecting at least one LV pacing site for an IMD equipped for cardiac stimulus pacing using a multi-pole lead according to an embodiment. The process 200, for example, may employ structures or aspects of various embodiments (e.g., systems and/or methods) discussed herein. For example, the process 200 may be performed using the IMD 100 shown in FIGS. 1 and 2. Optionally, an external programmer, such as external device 600 shown in FIG. 3, may be used in conjunction with the IMD 100 to perform the process 200. The process 200 is described herein with reference to the IMD 100, although other devices may be used instead of or in addition to the IMD 100. In various embodiments, certain aspects of the process 200 may be omitted or added, certain aspects may be combined, certain aspects may be performed simultaneously, certain aspects may be performed concurrently, certain aspects may be split into multiple aspects, certain aspects may be performed in a different order, or certain aspects or series of aspects may be re-performed in an iterative fashion.

In various embodiments, some portions, aspects, and/or variations of the process 200 may be able to be used as one or more algorithms to direct hardware to perform operations described herein. For example, the process 200 may be implemented in an algorithm that controls the IMD 100 to automatically select, program, and/or reprogram at least one LV pacing site according to the operations described herein. The process 200 may be performed in a clinic under the supervision of a clinician and/or in an unsupervised out-of-clinic setting. For example, the process 200 may suggest an LV pacing cathode to the clinician at implant of the IMD 100 and during in-clinic follow-ups. Out of the clinic, the process 200 may be performed to automatically reprogram the LV pacing cathode to a more preferred LV pacing site to produce an improved hemodynamic response to CRT.

At 202, a pacing pulse is delivered from at least one RV or RA pacing site. The pacing pulse is generated by the atrial pulse generator 170 and/or the ventricular pulse generator 172, depending on the pacing site selected. For example, the pacing site may be selected by the clinician or automatically by the microcontroller 160. As used herein, pacing site refers to the location of the cathode that is used to deliver a pacing pulse along a pacing vector. Example pacing pulses at an RV pacing site may be delivered at the RV tip electrode 132 or the RV ring electrode 134, along the pacing vectors RV tip 132 to RV coil 136 or RV ring 134 to RV coil 136, respectively. Optionally, the pacing vector may be unipolar between an RV cathode and the CAN 140. Example RA pacing sites may be at the atrial tip electrode 122 or the atrial ring electrode 123, along the pacing vectors from the respective electrodes 122, 123 to the SVC coil 138 or to the CAN 140. The pacing pulse may be delivered by the microcontroller 160 by sending a control signal 176, 178 to one or both pulse generators 170, 172 that identifies the pacing vector, the electrical output, the timing, and the like. The pulse generator 170 and/or 172 in response generates an electrical potential at one or both electrodes that define a selected pacing vector, resulting in a potential difference between the electrodes that induces a depolarization wave in the surrounding heart tissue.

At 204, LV activation events at multiple LV sensing sites are sensed. The LV activation events are generated in response to the pacing pulse. The LV activation events are detected responses to the propagating depolarization wave, and are detected along sensing vectors associated with corresponding LV sensing sites. As used herein, an LV sensing site refers to the location of an LV electrode that at least partially defines a sensing vector or channel over which the delivered pacing pulse is sensed. For example, the multiple LV sensing sites may be the locations of the LV electrodes, such as D1, M2, M3, and P4 of the quadripolar LV lead 124. In an embodiment, the IMD 100 senses along at least four sensing vectors, and each sensing vector utilizes a sensing electrode in the left ventricle. The sensing vectors associated with the LV sensing sites may be unipolar vectors D1-CAN, M2-CAN, M3-CAN, and P4-CAN. Therefore, in an embodiment, the pacing pulse is delivered at a RV pacing site and sensed at various LV sensing sites. This configuration may be referred to as RV pace—LV sense. Optionally, sensing vectors other than unipolar vectors may be used, such as D1-RV coil 136.

In an embodiment, the LV activation events are sensed by at least one sensor. For example, the sensor may be the ventricular sensing circuit 184, which includes an amplifier. For example, sensed electrical activity (e.g., voltage and/or current) at each electrode in a sensing vector may be routed as signals through the electrode configuration switch 174 to the ventricular sensing circuit 184. The ventricular sensing circuit 184 may amplify, convert, and/or digitize the received signals before forwarding the signals to the microcontroller 160 for recordation and analysis of the data. Optionally, various other sensors and/or sensing circuits may be used to sense the LV activation events instead of or in addition to the ventricular sensing circuit 184.

At 206, arrival times of the LV activation events for corresponding LV sensing sites are measured. The arrival times (e.g., conduction delays) each correspond to a conduction time from delivery of the pacing pulse until sensing of the corresponding LV activation event. For example, arrival times may be measured by recording the time that a pacing pulse is delivered at an RV site, recording the times of the LV activation events at each of the LV sensing sites, and subtracting the time of the pacing pulse from the times of the LV activation events. Optionally, the arrival times may be measured using an internal stopwatch by starting the time when the pacing pulse is delivered and stopping the time when each LV activation event occurs to determine the arrival time of each respective LV activation event. As an example, if it takes 60 ms after an RV pacing pulse for the sensor to detect an LV activation event at an LV sensing site, then the arrival time for that LV sensing site is 60 ms. The arrival times at the different LV sensing sites may vary due to the difference in locations of the LV electrodes relative to the cathode at the RV pacing site. Due to the different relative locations, the propagation wave may reach some LV sensing sites sooner than other sensing sites, resulting in a shorter arrival time.

The arrival times may be measured by the arrival measurement (AM) module 167 within the microcontroller 160. Optionally, the arrival times may be measured by the external programmer device 600. In such case, the IMD 100 may signal to the device 600 the time that a pacing pulse is delivered as well as the times for each of the LV activation events so the device 600 can determine the arrival times. For example, the device 600 may be a Merlin™ programmer (developed by St. Jude Medical, Inc.) with a freeze capture and calipers function capable of determining the time from the RV pacing marker to LV activation on each of the unipolar LV sensing vectors (e.g., D1-CAN, M2-CAN, M3-CAN, P4-CAN).

At 208, the differences between the arrival times for combinations of the LV sensing sites are calculated to obtain inter-site arrival delays between the combinations of the LV sensing sites. For example, inter-site arrival delay_{D1,M2} for combination D1, M2 may be calculated by subtracting the arrival time associated with the LV sensing site at the D1 electrode from the arrival time associated with the LV sensing site at the M2 electrode. Therefore, if the arrival time at sensing site D1 is measured to be 80 ms, and the arrival time at site M2 is 87 ms, the inter-site arrival delay_{D1,M2} would be the difference, 7 ms. The inter-site arrival delays may be calculated by the delay calculation (DC) module 168 within the microcontroller 160. Optionally, the external programmer device 600 may calculate the inter-site arrival delays for the various combinations of the LV sensing sites.

In an embodiment, inter-site arrival delays may be calculated for every available combination of the LV sensing sites. That is, the arrival time at each LV sensing site is subtracted from the arrival time at every other LV sensing site. For example, for LV electrodes D1, M2, M3, and P4, inter-site arrival delays may be computed for the six combinations: D1-M2, D1-M3, D1-P4, M2-M3, M2-P4, and M3-P4. Note that these example combinations represent pairings, not vectors, so the order of the electrodes in each pair is irrelevant. Optionally, when measuring the arrival times, the shortest arrival time from among the LV sensing sites may be determined. If the LV sensing site with the shortest or earliest arrival time is known, then only the differences between the arrival time at each of the LV sensing sites and the earliest arrival time need be calculated. Assuming, for example, that LV sensing site M3 is earliest with an arrival time of 67 ms, then the inter-site arrival delays need only be calculated for the combinations D1-M3, M2-M3, and P4-M3. Therefore, if the earliest arrival time is known, fewer inter-site arrival delays need to be calculated. In an embodiment, a clinician during implantation of the IMD 100 or during a follow-up may obtain inter-site arrival delays for all of the available combinations, or for at least the combinations between the arrival times of the LV sensing sites and the earliest measured arrival time. That way, the clinician can view how all of the arrival times for each of the LV sensing sites compare in order to determine one or more preferred LV pacing sites.

When the patient is out of the clinic, LV pacing therapy from a previously-selected LV sensing site may be currently taking place. At a designated time, the IMD 100 may automatically perform the process 200 to determine whether the current LV pacing site is still preferred. For example, the IMD 100 may perform process 200 to test whether the previously-selected LV site is still a preferred LV pacing site that will provide the best available acute hemodynamic response, or whether reprogramming the IMD 100 to pace from a different LV site would provide an improved acute hemodynamic response. In such situation, only the inter-site arrival delays between the arrival time at the current LV pacing site and the arrival times at the other LV sensing sites need be calculated. For example, if electrode M2 is the current LV pacing site, the combinations may be M2-D1, M2-M3, and M2-P4, and the pacing therapy will only be reprogrammed to deliver pacing from a different LV pacing site if another LV sensing site is preferable over the current site at the M2 electrode. Therefore, only the inter-site arrival delays that compare the other LV sensing sites to the current LV pacing site are desirable. Optionally, inter-site arrival delays may still be calculated for all available combinations of LV sensing sites, not just the combinations involving the current LV pacing site. The determination of whether one LV site would be preferable over another LV site is discussed herein below.

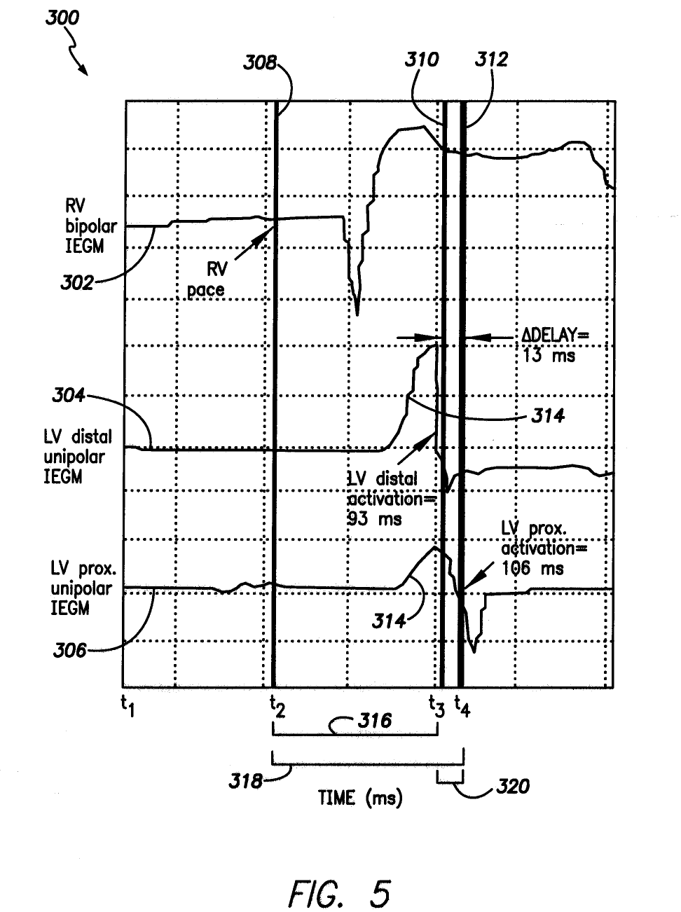

Referring now to FIG. 5, a graph 300 is displayed plotting multiple data streams measured in connection with different sensing sites. The data streams may be displayed on the graph 300 as intracardiac electrogram (IEGM) waveforms representative of the electrical activity in ventricular tissue (measured in mV) over time (measured in ms). The graph 300 displays an IEGM waveform 302 associated with an RV electrode and two IEGM waveforms 304, 306 associated with an LV distal electrode and an LV proximal electrode, respectively. For example, the RV electrode may be the RV tip 132 or RV ring 134, the LV distal electrode may be electrode D1 or M2, and the LV proximal electrode may be P4 or M3. The three IEGM waveforms 302-306 may be representative of the electrical activity sensed along sensing vectors at least partially defined by the respective corresponding electrodes. For example, waveform 302 may represent activity sensed along the bipolar RV vector RV tip 132 to RV coil 136, while the waveforms 304, 306 may represent activity sensed along unipolar LV vectors from the respective LV electrodes to the CAN.

The graph 300 displays the waveforms 302-306 vertically separated in order to compare the shape of the waveforms 302-306 over time. It should be recognized that the waveforms 302-306 share a common time scale but not the same electrical activity scale, meaning that the vertical distance between one waveform from the other waveforms does not represent a difference in the measured mV. The graph 300 may be a screenshot of a display (e.g., display 622 shown in FIG. 3) associated with programmer device 600 and/or IMD 100. Alternatively, the graph 300 may not be displayed, and is included herein for illustrative purposes to explain the internal process operations of the programmer device 600 and/or IMD 100.

The graph 300 illustrates how arrival times of LV activation events may be measured at operation 206 of process 200 (shown in FIG. 4) as well as how inter-site arrival delays may be calculated at operation 208 of process 200. For example, each of the sensing vectors may begin sensing for electrical activity at time t₁. At time t₂, an RV pacing pulse is delivered at an RV pacing site, which is denoted on the graph 300 by an RV pace marker 308. As the depolarization wave propagates through the myocardial tissue, the activity sensed at the RV and LV sensing sites are recorded in the waveforms 302-306. For example, the wave is used for biventricular (BiV) pacing, as the pulse delivered in the right ventricle propagates to the left ventricle where it is sensed at the LV sensing sites. The LV distal electrode waveform 304 indicates the presence of an LV activation event at time t₃, while the LV proximal electrode waveform 306 indicates an LV activation event afterwards at time t₄. The LV activation events for the LV distal electrode and the LV proximal electrode are denoted by activation event markers 310 and 312, respectively.

As shown in graph 300, the activation event markers 310, 312 represent the midpoint of the negative slope of the R-wave 314, which represents intrinsic ventricular depolarization, or the maximum negative slope of the R-wave 314. Optionally, the markers 310, 312 may be located at other locations along the R-waves 314 of the waveforms 304, 306, as long as the location selected is the same for both waveforms 304, 306, for comparison purposes. For example, the markers 310, 312 optionally may be located at the starting point of the R-wave 314 (e.g., leftmost location with a positive amplitude), the midpoint of the positive slope of the R-wave 314, the point of maximum positive slope, the apex of the R-wave 314, the nadir or lowest point of the R-wave 314, and the like. Optionally, the markers 310, 312 may be positioned manually by a clinician using a user interface to interact with the graph 300 on the display 622. Alternatively, markers 310, 312 may be positioned automatically, and the arrival times and inter-site arrival delays calculated automatically, by the IMD 100 and/or external programmer device 600 without assistance from a clinician or another third party. As such, the graph 300 may be purely a read-only and non-interactive graphic.

Once the LV activation events 310, 312 are determined, the arrival times for each of the LV sensing sites are computed by measuring the time between the time of the RV pacing pulse 308 and the times of the activation events 310, 312. For example, the LV activation event 310 for the LV distal electrode occurs at time t₃, the RV pacing pulse 308 occurs at time t₂, so the arrival time 316 for the LV sensing site at the LV distal electrode is the difference between times t₃ and t₂. As shown in FIG. 5, that difference was measured to be 93 ms, which represents the arrival time 316 of the LV activation event 310 at the LV distal sensing site. Likewise, the arrival time 318 for the LV activation event 312 at the LV proximal sensing site is represented as the time difference between the event 312 at time t₄ and the pacing pulse at time t₂, which is measured to be 106 ms. Since the arrival times 316, 318 for respective LV distal and proximal sensing sites are known, the inter-site arrival delay between these two sensing sites may be calculated as the difference between the two times 316, 318. For example, as shown in FIG. 5, the inter-site arrival delay (e.g., Δdelay) 320 for the combination of the LV distal sensing site and the LV proximal sensing site is 13 ms, calculated by subtracting 93 ms from 106 ms. The 13 ms delay 320 represents the time delay from time t₃ to time t₄.

Referring back to FIG. 4, at 210 a determination is made whether any of the inter-site arrival delays exceed a threshold, or delay cutoff. The threshold is a length of time, such as 10 ms. The threshold corresponds to a relative inter-site arrival delay between any pair of the LV sensing sites. Generally, if the inter-site arrival delay between a combination of two LV sensing sites exceeds the threshold, the LV site associated with the later of the two arrival times may result in an improved acute hemodynamic response if LV pacing is delivered at the later arriving LV site as opposed to pacing at the earlier arriving LV sensing site. On the other hand, if the inter-site arrival delay between the same combination of LV sensing sites is less than the designated threshold, then there may not be a significant difference in the acute hemodynamic response whether LV pacing is delivered at either of the LV sensing sites. Referring back to the example above, the inter-site arrival delay is 13 ms, which would exceed a threshold of 10 ms. The threshold may be programmable and capable of adjustment by the clinician at implant or during an in-clinic follow-up. Optionally, a default threshold value may be programmed into the microcontroller 160 of the IMD 100 prior to implant. If at least one of the inter-site arrival delays for a combination of LV sensing sites exceed the designated threshold, then flow moves along the branch denoted by “Y” to 212.

At 212, the LV sensing site from the corresponding combination that has a later arrival time is designated as a first LV pacing site from which to deliver LV pacing pulses. The first LV pacing site may correspond to one of the D1, M2, M3, or P4 electrodes provided on the multi-pole LV lead. The later arrival time is relative to the arrival times of other of the LV sensing sites. For example, when setting the IMD 100 in the clinic during implant or a follow-up, the later arrival time may be relative to the arrival times of all of the LV sensing sites, or at least relative to the earliest arrival time. On the other hand, when automatically testing to determine if the lacing site should be reprogrammed out-of-clinic, the later arrival time may be relative to the arrival time of the LV sensing site currently designated as the LV pacing site. The LV sensing site with the later arrival time is designated as the LV pacing site because, as stated above, pacing from this LV site may result in an improved acute hemodynamic response as opposed to pacing at the other LV site of the combination with the earlier arrival time. By designating the first pacing LV site, the IMD 100 may automatically program or reprogram to deliver future pacing sequences from the first LV pacing site. Additionally, or alternatively, the IMD 100 may be configured to provide a recommendation to a physician to use the LV sensing site having the later arrival time as the first LV pacing site. This recommendation may be useful in the clinic setting, where the physician/clinician is able to set the pacing parameters on-site based in part on the recommendations from the IMD 100. Referring back to 210, if on the other hand, none of the inter-site arrival delays for any combination of LV sensing sites exceed the designated threshold, flow moves along the branch denoted by “N” to 214.

At 214, since no calculated inter-site arrival delays exceed the threshold, the first LV pacing site is left unchanged. That is, assuming at least one LV pacing site had been previously selected to deliver LV pacing pulses during CRT, the IMD 100 would retain the previously designated LV pacing site as a designated site to deliver future LV pacing pulses for the time being. For example, since no inter-site arrival delays exceed the threshold, there are no LV sensing sites to which the patient may experience a significant improvement in acute hemodynamic response if pacing is delivered from one of those LV sites as opposed to continuing the pacing at the previously-selected LV pacing site. However, in the clinic setting there may be no previously designated LV pacing sites if the clinician is initially setting up the IMD 100 after implant. In such case, if none of the inter-site arrival delays exceeds the threshold, the IMD 100 and/or clinician will look to other factors besides arrival time to determine which LV pacing site should be designated. For example, the IMD 100 and/or clinician may look to available pacing vectors at each of the LV sensing sites, and designate the LV sensing site that has a pacing vector with a lowest capture threshold as the first LV pacing site. The local capture threshold is the minimum pacing output (e.g., energy) necessary to achieve local capture of the surrounding myocardial tissue along a particular pacing vector. In this case, the other factors, such as the local capture thresholds at pacing vectors, would be already determined and stored in the IMD 100.

After 212 or 214, a first LV pacing site should be designated. Once designated, future pacing pulses and/or sequences during pacing therapy may be delivered by the IMD 100 at the first LV pacing site. Optionally, pacing sequences during pacing therapy may be delivered from multiple LV pacing sites, but at least a first LV pacing pulse in the pacing sequence is delivered from the designated first LV pacing site. Furthermore, in an embodiment, if more than one LV sensing sites have a later arrival time that exceeds the threshold relative to the arrival time of the previously designated LV pacing site, for example, then multiple LV sensing sites may be designated as preferred LV pacing sites. For example, if the activation times measured after RV pacing for D1, M2, M3, and P4 were 180, 182, 195, and 197 ms, respectively, electrodes M3 and P4 would be presented as preferred cathodes based on their >10 ms arrival delay relative to D1 and M2. Optionally, the IMD 100 may look to other factors, such as pacing vector local capture thresholds, to determine a singular first LV pacing site out of the multiple preferred sites. On the other hand, and especially when performing multi-site LV pacing therapy using pacing sequences delivered from multiple LV pacing sites, the multiple preferred LV pacing sites may each be programmed to deliver pacing pulses of the pacing sequence.

Although one or more currently preferable LV pacing sites may be programmed through performing the process 200, during the course of CRT the electrical activation pattern may change as LV remodeling occurs. As a result, a previously-designated LV pacing site may no longer be preferable over one or more other LV sites. To keep the pacing at a preferred site out-of-clinic, the IMD 100 may be programmed to automatically perform the RV pacing to LV sensing measurements of process 200 at a frequency selected by the clinician. The frequency may be every day, every week, every two weeks, and the like.

FIG. 6 is a flow chart for a process 400 for selecting at least one LV pacing vector for an IMD equipped for cardiac stimulus pacing using a multi-pole lead according to an embodiment. The process 400 may be performed along with the process 200 shown in FIG. 4. More specifically, after calculating the difference between arrival times to obtain the inter-site arrival delays at 208, flow of the process 200 may proceed to process 400. Whereas process 200 designates an LV pacing site at which LV pacing is to be delivered, process 400 further determines one or more pacing vectors along which the pacing pulses are delivered. Like process 200, the process 400 may be performed using the IMD 100 shown in FIGS. 1 and 2, alone or in addition to the external programmer device 600 shown in FIG. 3. The process 400 may be performed by the IMD 100 according to an algorithm that is activated by a clinician in the out-of-clinic setting, where the IMD 100 implements the algorithm to automatically reprogram LV pacing to a preferred site, should the preferred site change (e.g., due to LV remodeling). Optionally, instead of automatically reprogramming, the IMD 100 may be configured to make a recommendation to a clinician for reprogramming post-implant or during a follow-up clinic visit. As such, the process 400 may be performed periodically (e.g., once every 1-14 days) after implant of the IMD 100 and after at least one LV pacing site has been programmed from which to deliver LV pacing pulses.

At 402, a determination is made as to whether at least one inter-site arrival delay exceeds a threshold. If not, flow proceeds along the branch denoted by “N” to 404. At 404, the current LV pacing site is left unchanged because it has not been determined that changing to another LV pacing site would make a significant acute hemodynamic response benefit over leaving the pacing site as is. If, however, at least one inter-site arrival delay does exceed the threshold, flow proceeds along the branch denoted by “Y” to 406.

At 406, the LV sensing sites from the corresponding combinations that have a later arrival time are designated as preferred LV pacing sites from which to deliver LV pacing pulses. There may be one or multiple such preferred LV pacing sites.

At 408, the available pacing vectors at the preferred sites are determined. For example, if the D1 electrode becomes a preferred pacing site, available pacing vectors at D1 include D1-M2, D1-P4, and D1-RV coil 136.

At 410, a determination is made as to whether at least one available pacing vector does not have phrenic nerve stimulation. Phrenic nerve stimulation (PNS) is a side effect of cardiac pacing that complicates the positioning of the LV lead. The phrenic nerve controls the diaphragm, and pacing pulses delivered too close to the phrenic nerve may stimulate the nerve, causing the patient's chest to intermittently “thump” or “pop.” In an embodiment, during algorithm setup, PNS information specific to various LV pacing vectors is entered into the IMD 100. Therefore, once the available pacing vectors are determined, the IMD 100 may be configured to determine whether any of those pacing vectors associated with the preferred LV pacing sites have PNS (e.g., would stimulate the phrenic nerve if a pacing pulse were delivered along the pacing vector). The IMD 100 may determine a phrenic nerve stimulation status of at least one pacing vector at each LV sensing site, and designate a pacing vector that has phrenic nerve stimulation as a non-pacing vector that is not to be used as a pacing vector at the first LV pacing site. If none of the available pacing vectors do not have PNS (e.g., all the available pacing vectors have PNS), all of the available pacing vectors are designated non-pacing vectors. As a result, flow moves along the branch denoted by “N” back to 404, where the current or previous LV pacing site is left unchanged. If, however, at least one available pacing vector does not have PNS, then flow of the process 400 moves along the branch denoted by “Y” to 412.

At 412, a determination is made as to whether at least one available pacing vector has a low local capture threshold, meaning a capture threshold that is below a predetermined value. For example, the predetermined value may be 3.5 V. Like PNS, the local capture thresholds for various pacing vectors may be determined and stored in the IMD 100 during algorithm set-up or at least prior to performing process 400. Although determined, the PNS status and local capture threshold of the available vectors may be re-tested and updated after implant during a follow-up visit to the clinic. All available pacing vectors, associated with the preferred LV pacing sites, that do not have PNS are evaluated to determine whether the local capture threshold for each pacing vector exceeds the predetermined value. Once the local capture thresholds of at least one pacing vector at each LV sensing site are determined, each pacing vector that has a high capture threshold (e.g., above the predetermined value) is designated as a non-pacing vector that is not to be used as a pacing vector at the first LV pacing site. If none of the available pacing vectors have a local capture threshold below the predetermined value, flow moves along the branch denoted by “N” back to 404, and the current or previous LV pacing site is left unchanged. If, however, at least one available pacing vector has a lower capture threshold than the predetermine value, flow of the process 400 moves along the branch denoted by “Y” to 414.

At 414, a vector from the available pacing vectors is selected as having no PNS and a low local capture threshold. If, for example, multiple available pacing vectors associated with a preferred LV pacing site do not have PNS and have low local capture thresholds, the IMD 100 may select each of the multiple pacing vectors. Optionally, the IMD 100 may select a single pacing vector from the multiple pacing vectors with the lowest local capture threshold and the absence of PNS. For example, out of the pacing vectors D1-M2, D1-P4, and D1-RV coil 136, mentioned above, preference may be given to the vector with the lowest capture threshold and absence of PNS.

At 416, the selected vector is programmed as a first LV pacing vector, along which LV pacing pulses during subsequent pacing therapy are delivered. The operations from 408 to 414 are intended to avoid switching to an LV pacing vector with a high capture threshold or PNS, which could each negatively affect the pacing therapy. As such, the algorithm may be configured to not switch to a vector with a capture threshold greater than 3.5 V, for example, or with PNS, even if the vector is associated with a preferred LV pacing site. As in 212 of process 200, once the first LV pacing vector is programmed, pacing pulses and/or sequences may be delivered along the first LV pacing vector. In an embodiment, after a designated period of time, such as between 1 and 14 days, the algorithm may implement processes 200 and 400 again to determine whether the current LV pacing vector is still the most preferable LV pacing vector.

One or more embodiments discussed herein describe determining an LV pacing site by, at least in part, measuring an RV pace—LV sense conduction delay (e.g., the arrival time of a propagating depolarization wave from an RV pacing site to an LV sensing site) at various LV sensing sites. In an alternate embodiment, a preferable LV pacing site may be determined using LV pace—RV sense measurements, such that an arrival time is measured between an LV pacing site and an RV sensing site. For example, multiple pacing pulses may be delivered sequentially or simultaneously along corresponding LV pacing vectors, where the LV electrodes D1, M2, M3, and P4 (shown in FIG. 1) are the cathodes in the LV pacing vectors and the anodes may be the CAN or other LV electrodes. The multiple pacing pulses are sensed at an RV sensing site, such as the RV coil 136 (shown in FIG. 1), and the respective arrival times are measured as the time from each LV pacing pulse to the activation event at the RV sensing site. The inter-site arrival delays may be calculated between the arrival times at a combination of LV pacing sites. For example, the IMD 100 may measure an arrival time of 60 ms from a pacing pulse delivered at LV pacing site D1 to be sensed at RV sensing site RV coil 136, and an arrival time of 71 ms from a different pacing pulse delivered at LV pacing site M3 to be sensed at the RV coil 136. The inter-site arrival delays between LV pacing sites D1 and M3 may be calculated as before, by subtracting 60 ms from 71 ms to get a delay_{D1,M3} of 11 ms. This arrival delay may be compared to a threshold to determine which LV pacing sites should be used to deliver further LV pacing pulses during pacing therapy. It should be noted that the threshold in alternate embodiments may be different than the threshold discussed above, since the arrival times are based on different measurements.

In an alternate embodiment, a preferable LV pacing site may be determined using RV sense—LV sense measurements, such that an arrival time is measured between an RV sensing site and an LV sensing site. In this embodiment, the pacing pulse that is sensed may be an intrinsic heartbeat of a patient. The heartbeat is sensed at an RV sensing site as well as at multiple LV sensing sites. Instead of measuring an arrival time from pace to sense, an RV-LV sensing delay may be measured for each LV sensing site, by determining the time delay between the activation event at the RV sensing site and the activation event at each respective LV sensing site. For example, the activation time at LV electrode M2 may be 8 ms after the activation time at the RV coil 136, while the activation time at LV electrode P4 is 15 ms after the RV coil 136. An inter-site arrival delay between LV sites may be calculated as the difference between RV-LV sensing delays, such as 7 ms for the delay between M2 and P4. As mentioned above, the inter-site arrival delays may be compared to a threshold, specific to the embodiment, to determine which LV sensing site would be a preferred site to deliver LV pacing pulses. Therefore, an extrinsic pacing pulse delivered by the IMD 100 need not be delivered in order to determine preferred LV pacing sites according to the embodiments described herein.

It is to be understood that the above description is intended to be illustrative, and not restrictive. For example, the above-described embodiments (and/or aspects thereof) may be used in combination with each other. In addition, many modifications may be made to adapt a particular situation or material to the subject matter of an embodiment described herein without departing from scope of the teachings herein. While the dimensions, types of materials and coatings described herein are intended to define parameters of one or more embodiments, they are by no means limiting and are exemplary embodiments. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A method for selecting at least one left ventricular (LV) pacing site for an implantable medical device equipped for cardiac stimulus pacing using a multi-pole LV lead, the method comprising:
sensing LV activation events at multiple LV sensing sites, where the activation events are generated in response to a delivery of a pacing pulse;
measuring arrival times of the LV activation events for the corresponding LV sensing sites, wherein the arrival times each correspond to a conduction time from delivery of the pacing pulse until sensing of the corresponding LV activation event;
calculating differences between the arrival times for combinations of the LV sensing sites to obtain inter-site arrival delays between the combinations of the LV sensing sites;
when at least one of the inter-site arrival delays exceeds a threshold, designating the LV sensing site from the corresponding combination that has a later arrival time as a first LV pacing site from which to deliver LV pacing pulses using the implantable medical device.

2. The method of claim 1, further comprising delivering a pacing sequence from the first LV pacing site.

3. The method of claim 1, further comprising delivering a pacing sequence from multiple LV pacing sites, wherein a first LV pacing pulse in the pacing sequence is delivered from the first LV pacing site.

4. The method of claim 1, wherein the sensing operation includes sensing along at least four sensing vectors, each sensing vector utilizing a sensing electrode in the left ventricle.

5. The method of claim 1, wherein the threshold corresponds to a relative inter-site arrival delay between any pair of the LV sensing sites.

6. The method of claim 1, wherein the later arrival time is relative to arrival times of other of the LV sensing sites.

7. The method of claim 1, further comprising providing a recommendation to a physician to use the LV sensing site having the later arrival time as the first LV pacing site.

8. The method of claim 1, wherein, when none of the inter-site arrival delays exceeds the threshold, retaining a previously designated LV pacing site as unchanged from which to deliver LV pacing pulses.

9. The method of claim 1, further comprising configuring the first LV pacing site to be a cathode within a pacing vector, the pacing vector extending between the first LV pacing site and at least one of a CAN electrode, a right atrial electrode, a right ventricular electrode, or an LV electrode at another LV sensing site.

10. The method of claim 1, wherein the first LV pacing site corresponds to one of a D1, M2, M3, or P4 electrode provided on the multi-pole LV lead.

11. The method of claim 1, further comprising determining a capture threshold of at least one pacing vector at each LV sensing site, and designating a pacing vector that has a high capture threshold as a non-pacing vector not to be used as a pacing vector at the first LV pacing site.

12. The method of claim 1, further comprising determining a phrenic nerve stimulation of at least one pacing vector at each LV sensing site, and designating a pacing vector that has phrenic nerve stimulation as a non-pacing vector not to be used as a pacing vector at the first LV pacing site.

13. The method of claim 1, wherein the pacing pulse is at least one of an intrinsic heartbeat or generated by the implantable medical device and delivered via the multi-pole LV lead.

14. The method of claim 1, wherein the pacing pulse is delivered using the implantable medical device from at least one right ventricular or right atrial pacing site.

15. An implantable medical device equipped for cardiac stimulus pacing using a multi-pole left ventricular (LV) lead, the device comprising:
a sensor configured to sense LV activation events at multiple LV sensing sites, where the activation events are generated in response to a delivery of a pacing pulse;
an arrival measurement (AM) module configured to measure arrival times of the LV activation events for the corresponding LV sensing sites, wherein the arrival times each correspond to a conduction time from delivery of the pacing pulse until sensing of the corresponding LV activation event;
a delay calculation (DC) module configured to calculate differences between the arrival times for combinations of the LV sensing sites to obtain inter-site arrival delays between the combinations of the LV sensing sites; and
a site designation (SD) module configured to, when at least one of the inter-site arrival delays exceeds a threshold, designate the LV sensing site from the corresponding combination that has a later arrival time as a first LV pacing site from which to deliver LV pacing pulses using the implantable medical device.

16. The implantable medical device of claim 15, further comprising a pulse generator configured to deliver a pacing sequence from the first LV pacing site designated by the SD module.

17. The implantable medical device of claim 15, further comprising a pulse generator configured to deliver a pacing sequence from multiple LV pacing sites, wherein a first LV pacing pulse in the pacing sequence is delivered from the first LV pacing site designated by the SD module.

18. The implantable medical device of claim 15, wherein the sensor senses along at least four sensing vectors, each sensing vector utilizing a sensing electrode in the left ventricle.

19. The implantable medical device of claim 15, wherein the DC module calculates a relative inter-site arrival delay between any pair of the LV sensing sites.

20. The implantable medical device of claim 15, wherein the DC module calculates the differences between the arrival time at each of the LV sensing sites and the arrival time at an LV sensing site previously designated as a current LV pacing site from which one or more LV pacing pulses have been delivered.

21. The implantable medical device of claim 15, wherein the DC module calculates the differences between the arrival time at each of the LV sensing sites and the earliest measured arrival time of an LV activation event generated in response to the pacing pulse.

22. The implantable medical device of claim 15, wherein, when none of the inter-site arrival delays exceeds the threshold, the SD module retains a previously designated LV pacing site as unchanged from which to deliver LV pacing pulses.

23. The implantable medical device of claim 15, wherein, when none of the inter-site arrival delays exceeds the threshold, the SD module designates the LV sensing site having a pacing vector with a lowest capture threshold as the first LV pacing site.

24. The implantable medical device of claim 15, wherein the first LV pacing site corresponds to one of a D1, M2, M3 or P4 electrode provided on the multi-pole LV lead.

25. The implantable medical device of claim 15, wherein the SD module obtains a capture threshold of at least one pacing vector at each LV sensing site, the SD module designating a pacing vector that has a capture threshold above a predetermined value as a non-pacing vector not to be used as a pacing vector at the first LV pacing site.

26. The implantable medical device of claim 15, wherein the inter-site arrival delay threshold is 10 ms.

27. The implantable medical device of claim 15, further comprising a pulse generator configured to deliver a pacing pulse from at least one right ventricular or right atrial pacing site.
